(19) United States
(12) Patent Application Publication
(10) Pub. No.: US 2018/0014633 A1
(43) Pub. Date: Jan. 18, 2018

(54) IONIC DEVICES

(71) Applicant: GLOBAL IONICS LLC, Ossining, NY (US)

(72) Inventor: Kenneth A. Davidov, Ossining, NY (US)

(73) Assignee: GLOBAL IONICS LLC, Ossining, NY (US)

(21) Appl. No.: 15/553,486

(22) PCT Filed: Feb. 25, 2016
(86) PCT No.: PCT/US16/19566

§ 371 Date: Aug. 24, 2017

Related U.S. Application Data
(60) Provisional application No. 62/120,403, filed on Feb. 25, 2015.

(51) Int. Cl. A46B 15/00; A61M 15/08; A61M 15/02; A23L 2/38; A61H 7/00; A23L 2/70; A61H 23/02

(52) U.S. Cl. CPC A46B 15/0024; A23L 2/70; A46B 15/0022; A61M 15/08; A61H 23/0236; A23L 2/38; A61H 7/005; A61M 15/02; A46B 2200/104; A61H 2201/0111; A61H 23/02

(57) ABSTRACT

A hair brushing or combing device, a nasal washing device, and a beverage ionizing device, comprising a metal anode being touched by the user; a cathode contacting with the liquid; an electronic circuit; and a switch operable to provide electrical potential to the metal anode, the switch also operable to cause activation of an ionic function.

### FIELD OF INVENTION

This application concerns ionic devices to provide a flow of ions for human use. In particular, a hair brush and comb, an ionic nasal rinse, and a beverage ionizing device are equipped to provide negative ions during usage.

### DESCRIPTION OF RELATED ART

Negative ions have been reported to have beneficial effects in many health aspects of a human body. For example, Dr. Svante August Arrhenius, a Swedish chemist who received the

Nobel Prize in Chemistry in 1903, discovered that negative ions are abundant near waterfalls and forests, causing the air to be fresher than elsewhere. At the turn of the 20^{th} century, Nobel Prize Winner in Physics, Dr. Phillip Eduard Anton Lennard, confirmed that negative ions are found in very high density in the basin of waterfalls where people feel especially refreshed and re-energized. Negative ions have been called “Vitamins of the air.” Among all the organs, our skin absorbs 85% of charged particles from the surroundings constantly. To human health, negative ions may strengthen the functions of autonomic nerves, reinforce collagen (tissues that are resilient and tension-related), improve the permeability of the cell's prototypical plasma membranes, and also strengthen the body's immune system.

Although the use of negative ions are known in other fields and have been reported to have beneficial effects on humans which includes feelings of relaxation, reduced tiredness, stress levels, irritability, depression and tenseness, no reports are believed to be published with regard to the effects of direct application of negative ions to the hair, the scalp, nasal walls & cavity, or for their use in beverage devices (e.g. a beverage bottle or beverage delivery device).

### SUMMARY OF THE DISCLOSURE

Disclosed is a smart hair brush and comb, with a circuit that may improve body health, by providing either or both ionic application and sonic vibrations to the scalp during usage on the scalp.

In another embodiment, an ionic nasal rinse can stimulate the nasal mucosa by providing ionic application to the inner walls of the of the nose and nasal cavity when the nasal rinse is used.

In another embodiment, a beverage ionizing device can providing ionic application to the body when the user drinks a beverage using the beverage ionizing device.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like.

The above and other objects, features, and advantages of various embodiments as set forth in the present disclosure will be more apparent from the following detailed description of embodiments taken in conjunction with the accompanying drawings.

### BRIEF DESCRIPTION OF THE DRAWINGS

FIG. 1A is an exploded view of a first embodiment of the present invention.

FIG. 1B is a perspective view of the first embodiment of the present invention as assembled.

FIG. 2A is another example of a first embodiment of the present invention.

FIG. 2B is a perspective view of another example of the first embodiment of the present invention.

FIG. 3 is a perspective view of a second embodiment of the present invention.

FIG. 4 is a perspective view of a first example of a third embodiment of the present invention.

FIG. 5 is a perspective view of a second example of the third embodiment of the present invention.

FIG. 6 is a perspective view of the second example of the third embodiment of the present invention when used by a user.

FIG. 7 is a perspective view of a third example of the third embodiment of the present invention.

FIG. 8 is a perspective view of a fourth example of the third embodiment of the present invention.

FIG. 9 is a perspective view of a fifth example of the third embodiment of the present invention.

FIG. 10 is a perspective view of a sixth example of the third embodiment of the present invention.

FIG. 11 is a perspective view of a seventh example of the third embodiment of the present invention.

FIG. 12 is a perspective view of a first example of a fourth embodiment of the present invention.

FIG. 13 is a perspective view of a second example of the fourth embodiment of the present invention.

FIG. 14A is a front view of a third example of the fourth embodiment of the present invention.

FIG. 14B is a vertical view of the third example of the fourth embodiment of the present invention.

### DETAILED DESCRIPTION

### First Embodiment

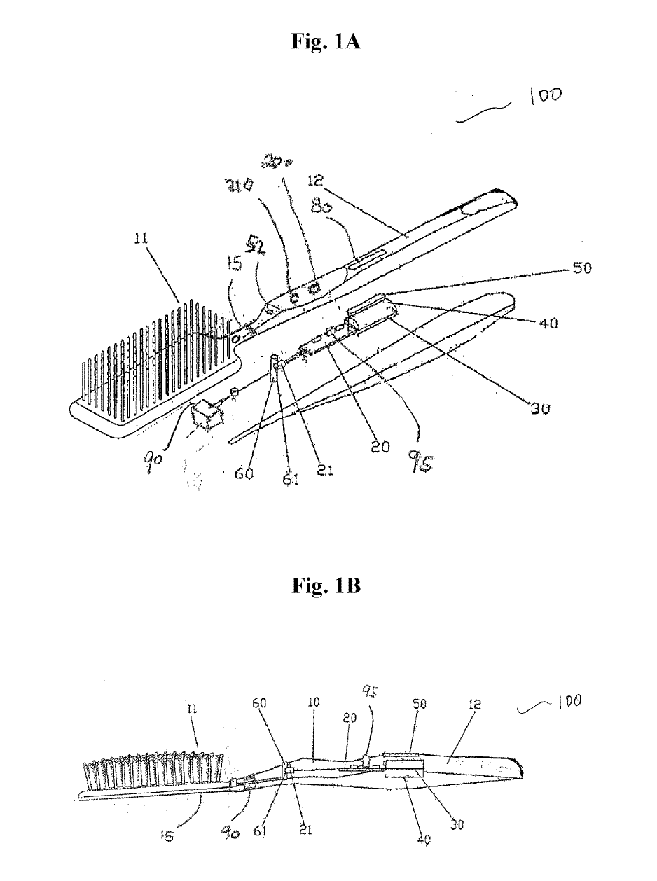

FIG. 1A-1B illustrate an example of an ionic brush 100 of the present invention. The ionic brush and comb 100 includes a handle portion 12 and a bristle brush end portion 15. A metal strip can act as an anode 50 and be positioned through an opening 80 in the handle 12 of the ionic brush and comb that a user's hand touches when the brush and comb is being used. The user's hand makes electrical contact with the anode and the user's hand can be wet or dry because the skin (hand) is naturally moist providing an electrical path for ions. Bristles or comb teeth 11 can conduct ions and may be referred to as an “ion emitter.” When in use, an electrical pathway for conducting ions is provided from bristles 11, though the user's scalp and body, and then to anode 50.

An electrical circuit 20 can be powered by one or more batteries 40 internal to the handle to provide electrical potential to anode 50. The battery or batteries can provide between 1.5V DC and 12V DC. Nominally, about 9V DC is the anticipated voltage level. A single 1.5V DC battery can be provided and the voltage boosted to a desired voltage, The battery or batteries can be housed in a holder 30. The electrical circuit has a positive terminal coupled to the anode, and a negative terminal coupled to the bristles. When the brush and comb is in use and the user is touching the metal strip 50, an electrical circuit is completed such that the electrical potential from the battery 40 coupled to bristles 11 causes ions to flow from the bristles to the metal strip 50 thereby causing the emitted ions at the bristles to be imparted to the surface of the user's scalp.

Body's energy (hand) starts the circuitry as a conductor, but also the body plays an role in the overall ionic function as the flow of negative ions from bristles of the brush (or teeth of the comb) from the bristles (ion emitter) through the scalp and back into the user's body or hand on the metal strip 50 to complete the electrical loop.

The bristle brush end portion 15 with bristles 11 is movable with respect to the handle 12. A vibrating device 90 can cause the bristles 11 to vibrate with respect to the handle 12. Such vibrating devices are well-known to those of ordinary skill. The bristle end 15 of the brush and comb may be removable and replaceable as is known in the art. Sonic or vibrating function usually provides 20,000 to 30,000 strokes or vibrations per minute. An ultrasonic brush function can be added and combined with ionic function or the user can use solely ultrasonic function or solely ionic function. A mode switch can be provided to effect the alternatives.

A mode switch 95 on the brush and comb handle is provided so the bristles 11 can be selected by the user to concurrently or alternatively vibrate with the emitting of ions. A light 60, which may be mounted on handle 12 through an opening 52 can be used to indicate when the ionic function is active. When the user combs or brushes their hair by using the ionic hair brush and comb, because the electrical loop is completed, the hairbrush and comb have the anti-static function. When the body controls is On, the hair and scalp will be stimulated by the ions that carry electric current generated by the battery power. This electric current stimulates the scalp and may be good for hair, strengthen hair roots, and refresh and increase memory. The combination of ionic and sonic functions in one hair brush and comb should provide the best results and performance for users.

A second switch 200 can have multiple settings to alter the number of vibrations per minute. A second light or lights 210, which may be mounted on handle 12, can be used to indicate which of the multiple settings the mode switch is in. The intensity of lights 210 can be used to indicate the relative level of ionic or sonic function.

One battery 40, for example an AAA or button battery, can be used to power the electrical circuit 20. The battery can be located within the handle and may be replaceable. Voltage booster circuitry (not shown) within the handle can boost 1.5 V up to about between 9 V to 12 V or any voltage necessary to activate the vibration device and provide ion flow. Alternatively, there can be multiple single cell batteries to power the hair brush and comb. The booster voltage 9V coupled with the natural resistance of the human skin and body is considered safe for the human body, and improve performance while ensuring safety.

The voltage booster circuit can generates 9 to 12 volts from a 1.5 volt AAA battery, which generates more negative ions (6× to 8×) and power during 1.5 to 2 minutes of brushing or combing. This elevated voltage can provide better results for stimulation to hair and scalp than at a lower voltage. The sonic-ionic brush and comb combines sonic vibration and 9 to 12 volts of ionic polarization power. The boosted voltage can do the work of 6 to 8 batteries in a small amount of time and in a small amount of space. Thus, the single cell 1.5 volt battery (coupled with the booster circuit) powers both sonic and ionic functions.

The user does not need to operate both ionic and sonic functions at same time. The user can use solely ionic function or solely sonic function, or both sonic and ionic together. The mode switch 95 as discussed above is provided for this purpose.

Alternatively, ionic function can operate using 1.5 V or 3 V and does not need to have a booster circuit for the ionic function. Although 9 V or 12 V is preferable to improve the ionic function and yield better results for stimulation to hair and scalp. The ionic function and/or sonic vibration may improve the delivery and/or function of grooming products applied to the hair. Additionally, such grooming products can be used in conjunction with the ionic brush or comb and symbiotically improve the ionic function.

A light 60 can turn on to show that ions are flowing and output is 9 V or 12 V (or an output that is required between 1.5 V and 12 V). This light can work at the same time sonic function is powered on or when user is solely using ionic function and sonic function is powered off. The light will glow to show the ionic function works when the user is holding the hair brush and comb and combing their hair. The user's body's energy starts the circuitry. In an implementation, the light 60 can include a light emitting device 21 electrically connected to the electronic circuit 20, and a light guide post 61 mounted to the handle located adjacent the light emitting device 21. The light guide post has a portion extending to the exterior the handle so as to transmit light from light emitting device. That is, the light source is visible through the light guide post and the light guiding and refracting material. In an implementation, light emitted by the light emitting device 21 is visible as shown in FIG. 1B on the same side of the handle as the bristles 11. In another implementation, the light emitted from the light emitting device 21 is visible on the opposite side of the handle on which the bristles are located. That is, the light is not directly visible to the user when the brush bristles are facing the user, as in when the user is combing their hair. Rather the light may be visible by reflection in a mirror in front of the user.

Bristles of the hair brush and comb are typically made of non-insulating materials, such as metal, conductive rubber, or conductive plastic, etc. If the hair brush and comb is designed for combing wet hair, because the wet hair and scalp is conductive, the bristles of hair brush and comb may be made of non-conductive materials.

The ionic function and power stimulates to hair and scalp easily, so users do not need to comb hard. However, users can comb at any level they wish. The sonic or ultrasonic hair brush function can have an ON/OFF switch 200. This switch may have multiple settings to offer options for speed and power. This switch may have a power light to indicate the ON position and a light or lights to indicate which of the multiple settings the device is in.

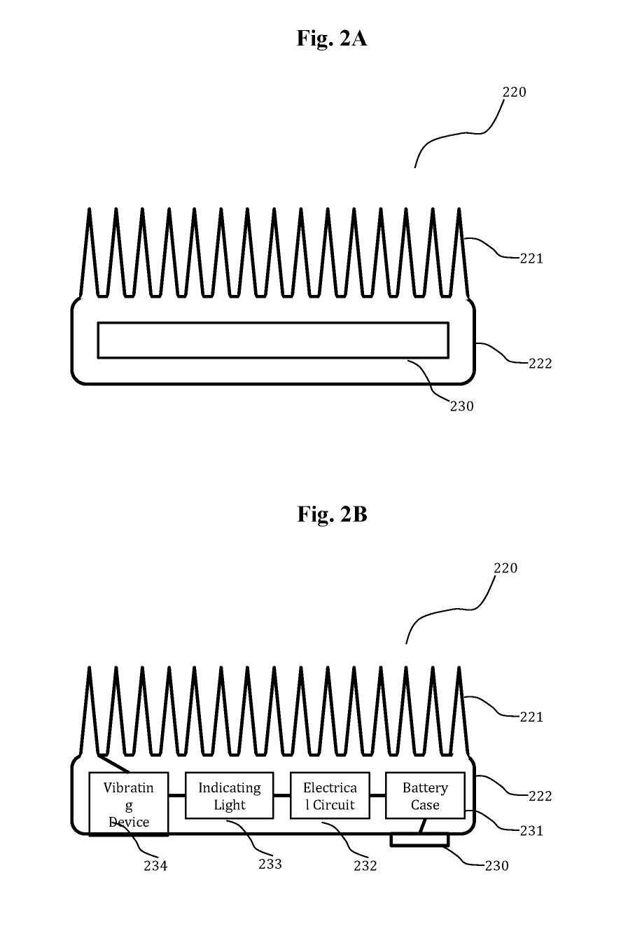

Alternatively, FIGS. 2A-2B illustrate an example of an ionic comb 220 of the present invention. As shown in FIG. 2A, the ionic comb 220 includes comb teeth 221 and a housing 222. In FIG. 2B, a conductive strip 230 that acts as an anode is positioned on the housing 222, so that when the comb is being used, a user's hand touches the conductive strip 230. The conductive strip 230 may be disposed on any surface of the housing 222 and can be of any desired length. The conductive strip 230 is connected with the electrical circuit 232, which is coupled to the indicating light 233. The indicating light 233 is connected to the vibrating device 234, which is coupled to the comb teeth 221, which can act as a cathode that conducts ions and may be referred to as an “ion emitter.” Similar with the ionic brush 100 shown in FIGS. 1A-1B, when the ionic comb is in use and the user is touching the conductive strip 230, an electrical pathway for conducting ions is provided from comb teeth 221, though the user's scalp and body, and then to anode 230.

In use the present hair brush and comb can be a disposable device or have a replaceable bristle head with soft polypropylene handle and conductive bristles. Despite the advanced function and benefits, the brush and comb can look and feel much like an ordinary brush and comb. The handle can be fully sealed to keep out water or substantially watertight and have a replaceable battery. The light can be a light-emitting diode (LED) or other lamp to indicate when the user has completed the electrical connection and the ionic action is functioning. The user may simply touch a finger or hand, preferably wet, to the metal anode on the handle while combing to engage the ionic action and see the light on the handle illuminate.

The sonic-ionic brush and comb will allow users to have an interactive combing experience. Users can comb with ionic polarization for 1 minute to stimulate the hair and scalp at 9 volts and also use sonic vibration for 1 minute to finish off with sonic power. Or users can comb with both sonic and ionic together, or sonic only. Users can make their own combing plan for what they feel is best suited for their hair and scalp and how they feel or want to feel.

Many bacteria, oil, dirt, and dry flaky skin on the scalp is positively charged, which can cause problems such as dandruff, folliculitis, and scalp acne. The sonic-ionic hair brush and comb emits negative ions to the scalp when the ionic function is on, which will temporarily change the polarity of the scalp to positive, loosen and repel the bacteria, oil, dirt, and flaky skin. Emitting negative ions along with the loop inside our body may yield the following typical negative ion benefits (see http://kiflow.com/info/ions.html):
Recovery from physical exhaustion or fatigue—by increasing oxygen levels in the blood and facilitating more efficient oxygen utilization, negative ions can help accelerate recovery from fatigue.
Stabilizing brain function—by promoting abundant oxygen levels in the blood, negative ions can help normalize brain function, resulting in relaxation and calmness.
Balancing the autonomic nervous system—negative ions can calm and relax taut nerves by balancing the opposing sympathetic and parasympathetic branches of the autonomic nervous system.
Cell rejuvenation—negative ions can help revitalize cell metabolism, enhancing the vitality of muscle tissue.

The sonic-ionic hair brush and comb technology can stimulate the hair and scalp with electric current, and provide refreshing relief.

### Second Embodiment

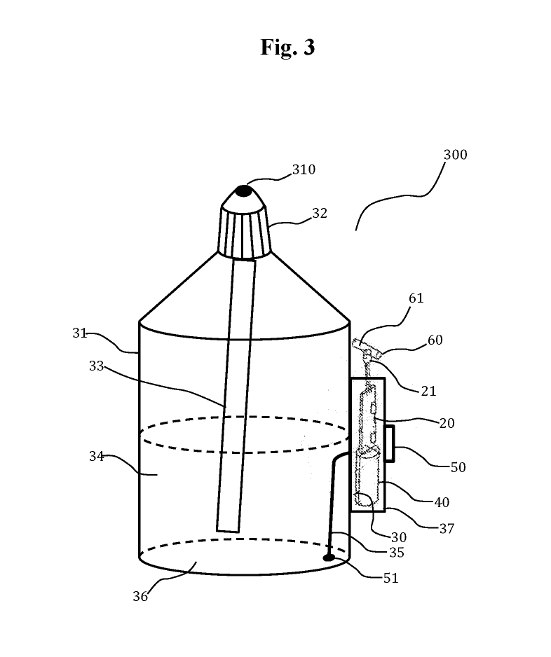

FIG. 3 illustrates an example of an ionic nasal rinse apparatus 300 of the present invention that includes a container 31, a cap 32 and a tube 33. The tube 33 is connected to the interior portion of the cap 32, to a hole 310 in the cap, and extended into the container 31. The cap 32 can be removed from the container 31 by rotating the cap 32, for example, counter-clockwise, so that the container 30 may be filled with saline solution 34. A battery case 37 is fixed to the container 31. A conductive strip 50, which acts as an anode, is positioned on the battery case 37 and touched by a user hand when the nasal rinse container is being used. The user's hand can be wet or dry because the skin (hand) is naturally moist providing an electrical path for ions. A conductive cathode 51, also referred herein as “ion emitter”, is connected to the battery case 37 through conductive wire 35, which is extended into the container 31 and located adjacent the bottom 36 of the container 31. The wire 35 may be flexible food safe and can be insulated in part with, for example, a soft or hard plastic.

The battery case 37 includes an electrical circuit 20 powered by one or more batteries 40 that provides electrical potential to the anode. The battery or batteries can be housed in a holder 30. The battery or batteries can provide between 1.5V DC and 12V DC. Nominally, about 9V DC is the anticipated voltage level. A single 1.5V DC battery can be provided and the voltage boosted to a desired voltage. One battery 40, for example an AAA or button battery, can be used to power the electrical circuit 20. The battery can be located within the battery case and be replaceable. Voltage booster circuitry (not shown) within the battery case can boost 1.5 V up to about between 9 V to 12 V or any voltage necessary to provide ion flow. Alternatively, there can be multiple single cell batteries to provide ions to the nasal rinse.

In use, the user holds the nasal rinse 300 and touches the conductive strip 50, the body's energy (hand) starts the circuitry as a conductor. As is known in nasal rinses, the user puts the cap 32 in a nostril and squeezes the container 31 to cause saline solution 34 to be ejected through hole 310 and into the user's nostril, The user's body also plays a role in the overall ionic function as the flow of negative ions from the cathode (ion emitter) of the nasal rinse through the saline solution 34 into the user's nose and back into the user's body or hand on the conductive strip 50 to complete the electrical loop.

Because the nasal rinse contains salts such as Sodium Chloride (NaCl) and Sodium Bicarbonate (NaHCO₃), the saline solution 34 may be sodium water with a lot of free negative ions (Cl⁻ and HCO₃⁻) and positive ions (Na⁺). Because the wire 35 is positioned inside the saline solution 34, attached to the battery and connected to cathode 51, the boosted voltage 9V is added to the saline solution 34. The wire 35 will attract and consume positive ions, i.e., the wire functions like a negative ion emitter, which leaves more negative ions in the saline solution 34, so that the negative ions of the saline solution is increased.

When the nasal rinse is in use, the user's hand is touching the conductive strip 50 and the saline solution 34 flows into the user's nose, an electrical circuit is completed such that the electrical potential from the battery 40 at the cathode 51 causes ions to flow from the cathode to the metal strip 50 thereby causing the emitted ions to be imparted to the surface of the user's nose and in to the nasal cavity through the saline solution 34 flowing into the nose.

A light 60, which may be connected with the battery case 37 and mounted on the container 31, can be used to indicate when the ionic function is active. When the user is using the nasal rinse, or even just squeezing the saline solution 34 on the body, for example, on the hand, because the electrical loop is completed, the light 60 will be on. Using the ionic nasal rinse can temporarily change the internal surfaces of nasal and sinus passages to positive charge and loosens and repel the mucus, and the this effect will be enhanced when there is higher voltage generated by the booster circuitry, or there is higher density of free negative ions in the saline solution 34.

The voltage booster circuit 20 can generates 9 to 12 volts from a 1.5 volt AAA battery, which generates more negative ions (6× to 8×) and power during 1.5 to 2 minutes of using. This elevated voltage can provide better results for using the nasal rinse than at a lower voltage.

Alternatively, ionic function can operate using 1.5 V or 3 V and does not need to have booster circuit for the ionic function. Although 9 V or 12 V is preferable to improve the ionic function and yield better results for use.

In an implementation, the light 60 can include a light emitting device 21 electrically connected to the electronic circuit 20, and a light guide post 61 mounted to the container 31 and located adjacent the light emitting device 21. The light guide post has a portion extending to the exterior the container so as to transmit light from light emitting device. That is, the light source is visible through the light guide post and the light guiding and refracting material.

The battery case 37 can be fully sealed to keep out water or substantially watertight and have a replaceable battery. The light can be a light-emitting diode (LED) or other lamp to indicate when the user has completed the electrical connection and the ionic action is functioning.

Therefore, when the nasal rinse is used, the user fills the plastic cup with warm, distilled water is preferred, but clean or filtered tap water can be used, and pour in a premixed saline packet and shake the mixture. When the user's hand touches the metal conductive strip 50 on the side of the plastic container 31, and the user squeezes the plastic container 31 when the tip of the cap 32 touches the nose, an electronic circuit is formed, comprising the human body, a voltage booster circuit, and a single AAA battery inside the waterproof case. Through electrochemical reactions within the saline mixture, the number of negative ions from the ionic emitter are greatly enhanced. Millions of negative ions through the power of water (at 9 volts) enter the nasal cavity.

The walls of the sinuses are negatively charged and mucus is positively charged. As opposites, they attract. The nasal rinse temporarily changes the sinus walls to a positive charge and loosens and repels the mucus. Also, the flow of negative ions can polarize many types of bacteria and cause the bacteria to become inactive. At the same time, the polarization effect keeps the sinuses feeling clean and clear. Thus, the ionic nasal rinse can stimulate the nasal mucosa through anion or micro-electric current (9V DC power), which can have an anti-allergic effect for people suffering or experiencing symptoms from rhinitis. Alternatively, a solution with medicine may be a substitute for the saline solution for conditions that require a medicine to be administered through the nose and/or nasal cavity.

### Third Embodiment

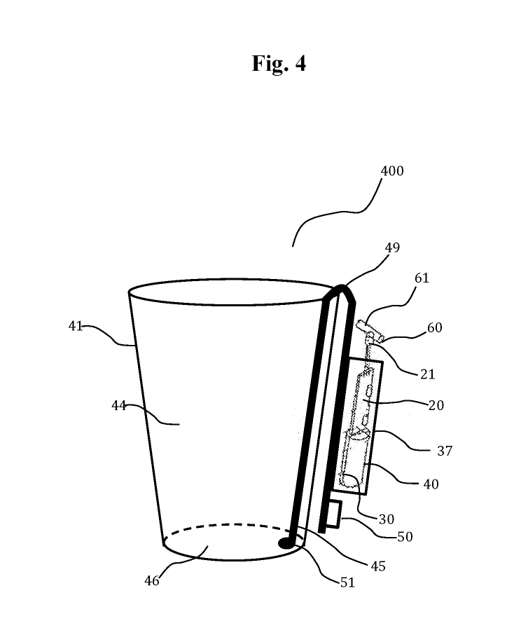

FIGS. 4-6 illustrate examples of a beverage ionizing device of the present invention.

As shown in FIG. 4, a beverage ionizing device 400 includes a container 41 which may be filled with beverage 44. The beverage 44 may be juices, sports drinks, coffee, tea, beer, wine, medicine, etc. The container 41 may be a cup, a bottle, a pitcher, a flask, etc and made of materials such as plastic, glass, metal, ceramic, etc. The beverage ionizing device 400 may further include a cap (not shown) covering the container 41, and a straw (not shown) extended into the container 41. A removable unit 49 is mounted on the container 41. The removable unit 49 includes a battery case 37, wire 45, conductive strip 50, a conductive cathode 51, and a light 60. The battery case 37 is fixed to the wire 45, which is extended into the container 41 and located adjacent the bottom 46 of the container 41. The wire 35 may be flexible food safe and can be insulated in part with, for example, a soft or hard plastic or another conductive material. The conductive cathode 51 is connected to the battery case 37 through the wire 45. The conductive strip 50, which is can be a metal strip, acts as an anode, is also connected to the battery case 37 through the wire 45. The conductive strip 50 is touched by a user hand when the user is holding the beverage ionizing device. The user's hand can be wet or dry because the skin (hand) is naturally moist providing an electrical path for ions. The light 60 may be connected with the battery case 37, or connected to the wire 45 directly. The removable unit 49 may be a one-piece unit or a multi-piece unit. The removable unit 49 is removable from the container 41, which can make the container more portable and easier to clean.

The battery case 37 includes an electrical circuit 20 powered by one or more batteries 40 that provides electrical potential to the anode. The battery or batteries can be housed in a holder 30. The battery or batteries can provide between 1.5V DC and 12V DC. Nominally, about 9V DC is the anticipated voltage level. A single 1.5V DC battery can be provided and the voltage boosted to a desired voltage. One battery 40, for example an AAA battery, can be used to power the electrical circuit 20. The battery can be located within the battery case and be replaceable. Voltage booster circuitry (not shown) within the battery case can boost 1.5 V up to about between 9 V to 12 V or any voltage necessary to provide ion flow. Alternatively, there can be multiple single cell batteries to power the beverage ionizing device.

When the user hold the beverage ionizing device 400 and touches the conductive strip 50, the body's energy (hand) starts the circuitry as a conductor, and the body also plays a role in the overall ionic polarization function as the flow of negative ions from the cathode (ion emitter) of the beverage ionizing device through the beverage 44 to the user's mouth and back into the user's body or hand on the conductive strip 50 to complete the electrical loop.

The beverage 44 may be a sports drink, which is good for rehydrating the body as known. When the electrical loop is on, many negative ions are added into the water, which may slightly raise the pH level of the beverage. The beverage 44 may also be juice and flavored water beverages which include abundant negative ions that are good for body. Since the wire 45 is positioned inside the beverage 44, attached to the battery and connected to cathode 51, the boosted voltage 9V is added to the beverage 44. The wire 45 will attract and consume positive ions, i.e., the wire functions like a negative ions emitter, which leaves more negative ions in the beverage 44, so that the beverage 44 is full of negative ions.

When the beverage ionizing device is in use, the user's hand is touching the conductive strip 50 and the user is drinking (the user lip and mouth can make contact with the beverage ionizing device) the beverage 44 from the container 41, an electrical circuit is completed such that the electrical potential from the battery 40 at the cathode 51 causes ions to flow from the cathode to the conductive strip 50 thereby causing the emitted ions to be imparted to the surface of the user's lip and mouth through the beverage 44 flowing into the mouth.

Therefore, in order to increase the number of negative ions in the beverage 44, since the wire 45 in the beverage 44 is connected to cathode 51, this wire is like a negative ions emitter. This ions emitter also consumes positive ions inside the beverage 44, which leaves more negative ions in the beverage 44.

The light 60 can be used to indicate when the ionic polarization function is active. When the user is using the beverage ionizing device, because the electrical loop is completed, the light 60 will be on. Using the beverage ionizing device will not only ionize the beverage and add voltage (for example, 9V), but also generate millions of negative ions that can speed up recovery due to dehydration, boost energy, flush out toxins, act as an antioxidant, and enhance the body's immune system. These effects will be enhanced when there is higher voltage generated by the booster circuitry, or there is higher density of free negative ions in the beverage 44.

The voltage booster circuit 20 can generate 9 to 12 volts from a 1.5 volt AAA battery, which generates more negative ions (6× to 8×) and power during usage of the beverage ionizing device. This elevated voltage can provide better results for using the beverage ionizing device than at a lower voltage.

Alternatively, ionic polarization function can operate using 1.5 V or 3 V and does not need to have booster circuit for the ionic function. Although 9 V or 12 V is preferable to improve the ionic function and yield better results for use.

In an implementation, the light 60 can include a light emitting device 21 electrically connected to the electronic circuit 20, and a light guide post 61 mounted to the container 41 and located adjacent the light emitting device 21. The light guide post has a portion extending to the exterior the container so as to transmit light from light emitting device. That is, the light source is visible through the light guide post and the light guiding and refracting material.

The battery case 37 can be fully sealed to keep out water or substantially watertight and have a replaceable battery. The light can be a light-emitting diode (LED) or other lamp to indicate when the user has completed the electrical connection and the ionic action is functioning.

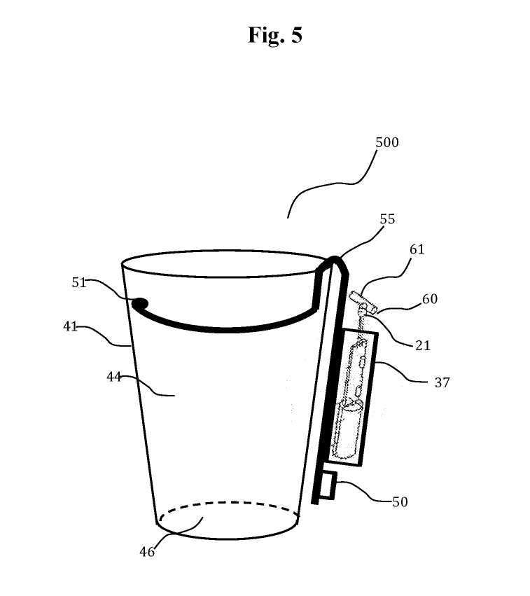

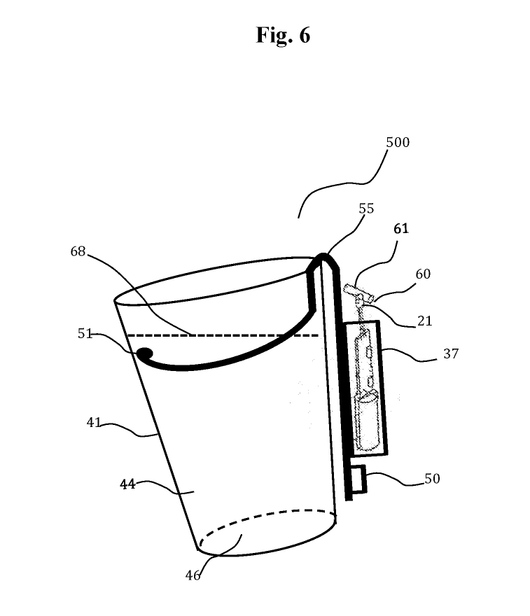

Alternatively, FIG. 5 illustrates another example of the beverage ionizing device 500. As shown in FIG. 5, the wire 55 is curved along the wall of the container 41. Therefore, when the user is drinking the beverage 44 and the beverage ionizing device 500 is tilted, as shown in FIG. 6, the surface of the liquid is shown as liquid level 68, so that the curved wire 55 is always contacting with the liquid when the user is drinking, and the electric loop is completed all the time during drinking.

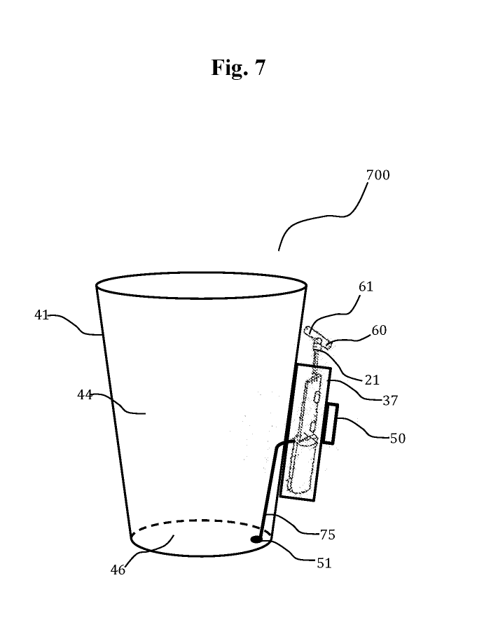

FIG. 7 illustrates another example of the beverage ionizing device 700. The battery case 37 is fixed to the container 41 directly. The conductive strip 50 may be positioned on the battery case 37. The metal cathode 51 is connected to the battery case 37 through wire 75, which is extended into the container 41 and located adjacent the bottom 46 of the container 41. The light 60 is connected to the battery case 37 and mounted on the container 41. Thus, as shown in FIG. 7, the battery case 37, the wire 75, the conductive strip 50, the conductive cathode 51 and the light 60 comprise a permanent unit fixed to the container 41.

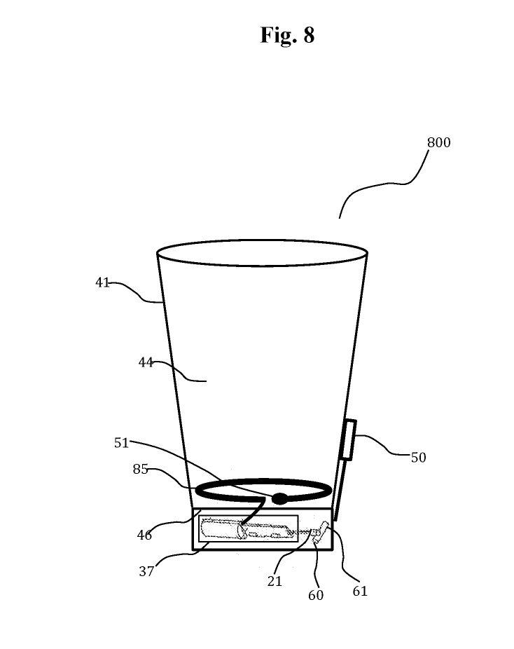

FIG. 8 illustrates another example of the beverage ionizing device 800. The battery case 37 is fixed to the bottom 46 of the container 41 directly. The conductive strip 50 may be positioned on the wall of the container 41 and connected to the battery case 37. The conductive cathode 51 is connected to the battery case 37 through wire 85. The wire 85 is extended into the container 41, located adjacent the bottom 46, and curved along the wall of the container 41. Therefore, when the user is drinking the beverage 44, even if when the beverage ionizing device 800 is tilting, the curved wire 85 is always contacting with the liquid, and the electric loop is completed all the time when the user is drinking. The light 60 is connected to the battery case 37 and located on the bottom 46 of the container 41. Thus, as shown in FIG. 8, the battery case 37, the wire 85, the conductive strip 50, the conductive cathode 51 and the light 60 comprise a permanent unit fixed to the bottom 46 of the container 41.

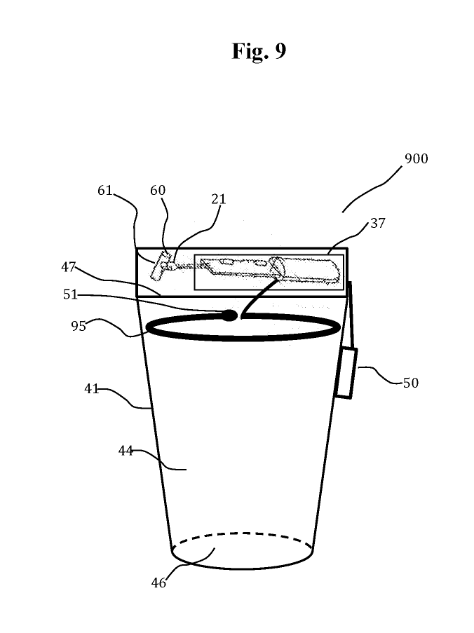

FIG. 9 illustrates another example of the beverage ionizing device 900. The battery case 37 is fixed to the lid 47 of the container 41 directly. The conductive strip 50 may be positioned on the wall of the container 41 and connected to the battery case 37. The metal cathode 51 is connected to the battery case 37 through wire 95, which is extended into the container 41, located adjacent the lid 47, and curved along the wall of the container 41. Therefore, when the user is drinking the beverage 44, even when the beverage ionizing device 900 is tilted, the curved wire 95 is always contacting with the liquid, and the electric loop is completed all the time when the user is drinking. The light 60 is connected to the battery case 37 and located on the lid 47 of the container 41. Thus, as shown in FIG. 9, the battery case 37, the wire 95, the conductive strip 50, the conductive cathode 51 and the light 60 comprise a permanent unit fixed to the lid 47 of the container 41.

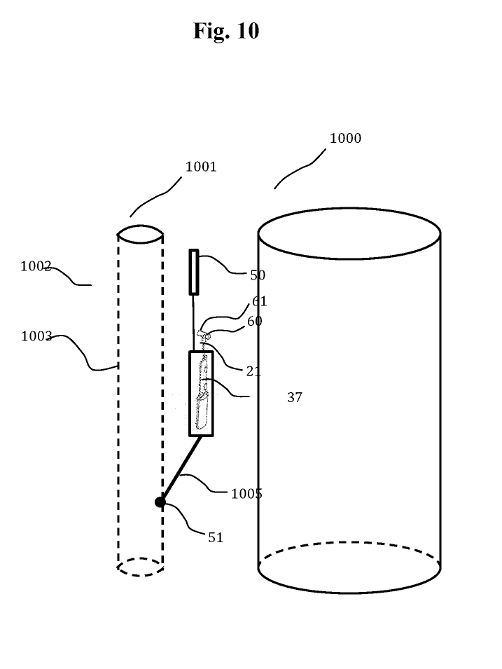

FIG. 10 illustrates another example of the beverage ionizing device 1000, which is a beverage ionizing straw that can be used with any container for the user to drink a beverage. The tube 1001 can be made of non-conductive material. The battery case 37 is fixed to the outer surface 1002 of the tube 1001. The conductive strip 50 may be positioned on the upper part of the outter surface 1002 of the tube 1001, so that the conductive strip 50 may be touched by the user's lip when the user is sipping the beverage through the tube 1001. The conductive cathode 51 is located on the inner surface 1003 of the tube 1001 and connected to the battery case 37 through wire 1005. Therefore, when the user is drinking the beverage 44 through the tube 1001, the conductive cathode 51 is always contacting the beverage, and the electric loop is completed all the time when the user is drinking. The light 60 is connected to the battery case 37 and located on the outer surface 1002 of the tube 1001. Thus, as shown in FIG. 10, the battery case 37, the wire 1005, the metal conductive strip 50, the metal cathode 51 and the light 60 comprise a permanent unit fixed to the tube 1001.

When the user drinks the beverage using the beverage ionizing device 1000 shown in FIG. 10, the user's lip touches the conductive strip 50 on the tube 1001 and sips the beverage 44 through the straw, which completes an electrical circuit, such that the electrical potential from the battery 40 at the cathode 51 causes ions to flow from the cathode (ion emitter) through the beverage 44 to the user's mouth and then back on the conductive strip 50 to complete the electrical loop.

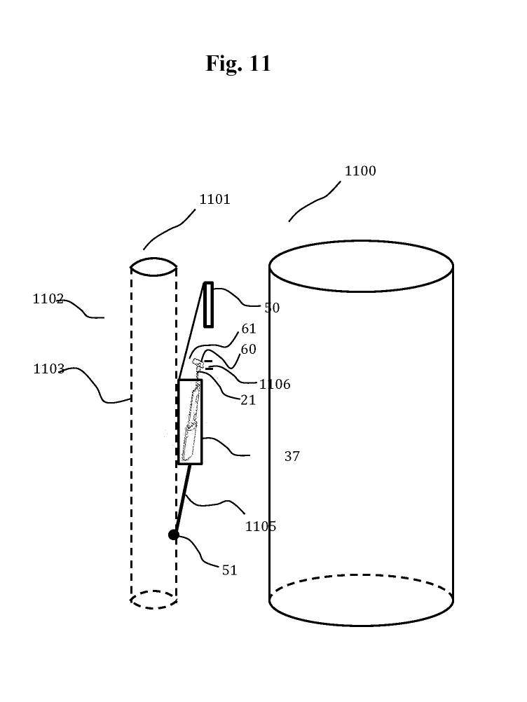

Alternatively, FIG. 11 illustrates another example of the beverage ionizing device 1100, which is similar with the beverage ionizing device 1000 shown in FIG. 10, except that the battery case 37 and light 60 are located inside the tube. In the beverage ionizing straw 1100, the tube 1101 can be made of non-conductive material. The battery case 37, and the light 60, which is connected to the battery case 37, are located in the tube 1101, for example between the inner surface 1103 and the outer surface 1102. When the light 60 is on, the light may be seen through an opening 1106 on the outer surface 1102 of the tube 1101. The conductive strip 50 may be positioned on the upper part of the outer surface 1102 of the tube 1101, so that the conductive strip 50 may be touched by the user's lip when the user is sipping the beverage through the tube 1101. The conductive cathode 51 is located on the inner surface 1103 of the tube 1101 and connected to the battery case 37 through wire 1105. Therefore, when the user is drinking the beverage 44 through the tube 1101, the metal cathode 51 is always contacting with the beverage, and the electric loop is completed all the time when the user is drinking. Thus, as shown in FIG. 11, the battery case 37, the wire 1105, the conductive strip 50, the metal cathode 51 and the light 60 comprise a permanent unit fixed to the tube 1101.

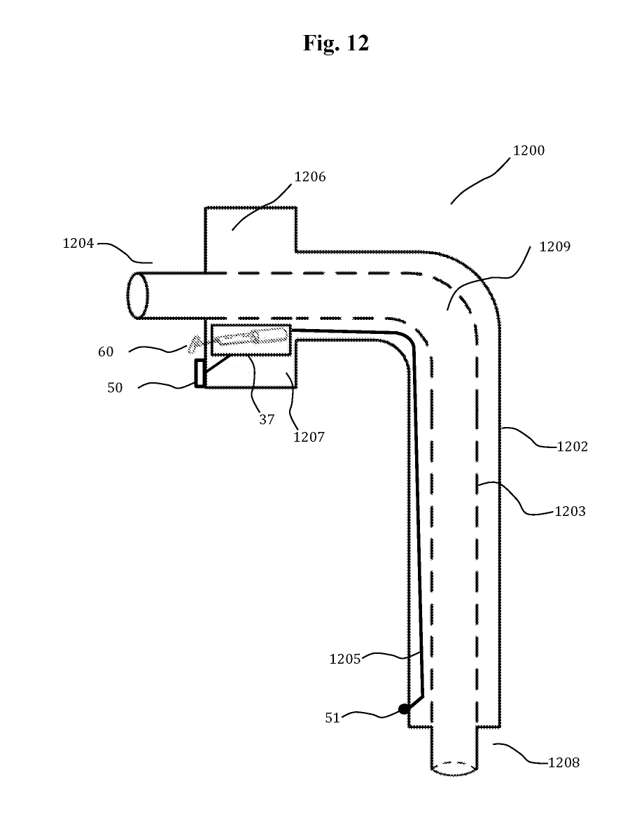

FIG. 12 illustrates another example of the beverage ionizing device 1200, which is also a beverage ionizing straw that can be used with any container for the user to drink beverage. The beverage ionizing device 1200 has two protrusions 1206 and 1207 adjacent to the top 1204 of the device 1200, so that the beverage ionizing device 1200 can rest on/over the edge of a cup, a bottle, or any other beverage containers. Alternatively, the protrusion may be a protruding collar surrounding the top 1204 of the beverage ionizing device 1200 so that the device 1200 may rest on the beverage container in any direction.

As in FIG. 12, the battery case 37 is located inside the beverage ionizing device 1200, for example, in the protrusion 1207 and between the inner surface 1203 and the outer surface 1202. The light 60, which is connected to the battery case 37, may be located outside the protrusion 1207 as shown in FIG. 12. Alternatively, the light may be located inside the protrusion 1207, and when the light 60 is on, the light may be seen through an opening (not shown) on the outer surface of the protrusion 1207.

The conductive strip 50 may be positioned on the outer surface of the protrusion 1207 so that the user may press thumb or finger on the conductive strip 50 when sipping the beverage using the beverage ionizing device 1200. The conductive cathode 51 is located on the lower portion of the outer surface 1202 and connected to the battery case 37 through wire 1205. Alternatively, the conductive cathode 51 may be located on the inner surface 1203. Therefore, when the user is drinking the beverage through the device 1200, the conductive cathode 51 is always contacting with the beverage, and the electric loop is completed all the time when the user is drinking.

As shown in FIG. 12, the diameter of the beverage ionizing device 1200 may be smaller at the bottom 1208. The beverage ionizing device 1200 is curved and may be made of any flexible material, for example, plastic, metal, rubber, etc. or have a flexible section for bending a portion of the straw-like beverage ionizing device at a particular location. The beverage ionizing device 1200 can be made in assorted sizes for smaller cups or even a very small container for medicine or other heath applications. The beverage ionizing device 1200 may be disposable, or have replaceable battery case 37. The beverage ionizing device is easy to clean by running water and soap through the hollow portion 1209 in the center of the device 1200. The straw can be thrown away and replaced.

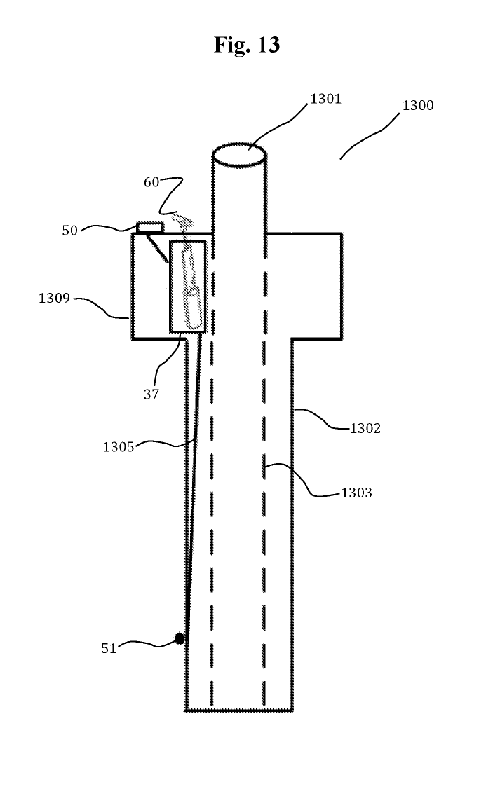

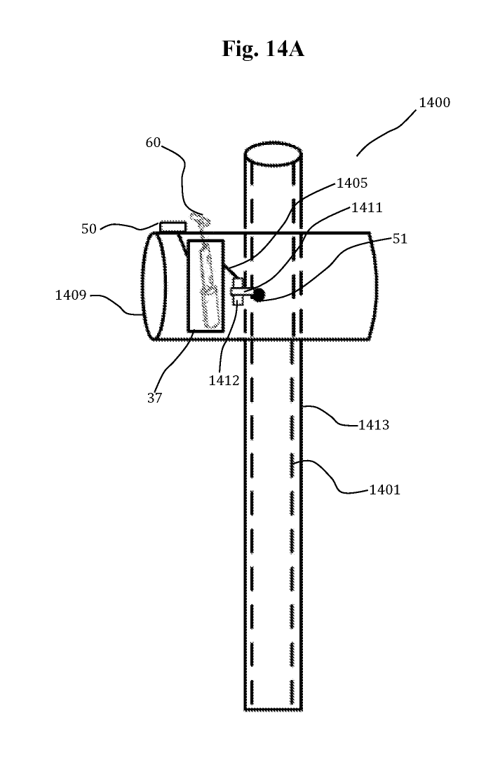

Alternatively, FIGS. 13 illustrate another example of the beverage ionizing device 1300, which is similar with the beverage ionizing device 1200, except that the tube 1301 is straight, and the beverage ionizing device 1300 can rest on/over the edge of a cup, a bottle, or any other beverage containers using the protrusion 1309.

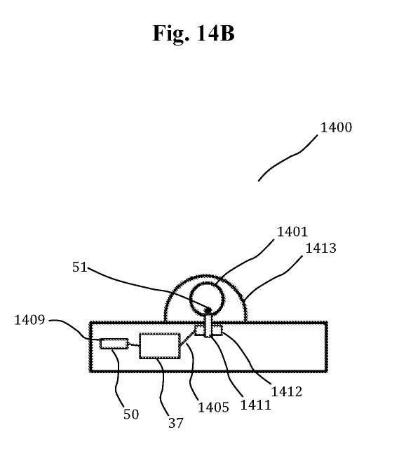

FIGS. 14A-14B illustrate another example of the beverage ionizing device 1400, which is also a beverage ionizing straw that can be used with any container for the user to drink beverage. FIG. 14A illustrates a front view of the beverage ionizing device 1400. The beverage ionizing device 1400 includes a tube 1401 inserted in a tube sleeve 1413, and a protrusion 1409 connected with the tube sleeve 1413, so that the beverage ionizing device 1400 can rest on/over the edge of a cup, a bottle, or any other beverage containers. Alternatively, the protrusion 1409 may be a protruding collar surrounding the tube 1401 so that the device 1400 may rest on the beverage container in any direction.

The battery case 37 is located inside the protrusion 1409. The light 60, which is connected to the battery case 37, may be located outside the protrusion 1409 as shown in FIG. 14A. Alternatively, the light may be located inside the protrusion 1409, and when the light 60 is on, the light may be seen through an opening (not shown) on the outer surface of the protrusion 1409. The conductive strip 50 may be positioned on the outer surface of the protrusion 1409 and connected with the battery case 37 so that the user may press thumb or finger on the conductive strip 50 when sipping the beverage using the beverage ionizing device 1400.

FIG. 14B illustrates a vertical view of the beverage ionizing device 1400. When the tube 1401 is inserted in the tube sleeve 1413, a tube contact portion 1411 on the tube 1401 will be connected with the sleeve contact portion 1412 on the protrusion 1409, and the sleeve contact portion 1412 is connected to the battery case 37 through wire 1405, so that the conductive cathode 51, which is located on the tube contact portion 1411 will be connected to the battery case 37. Therefore, when the user drinks the beverage using the beverage ionizing device 1400, the user's hand touches the conductive strip 50 and sips the beverage through the straw, which completes an electrical circuit, such that the electrical potential from the battery at the cathode 51 causes ions to flow from the cathode (ion emitter) through the beverage to the user's mouth and hand, and then back on the conductive strip 50 to complete the electrical loop.

The beverage ionizing device 1400 is waterproof, easy to produce, and easy to replace the battery. The tube 1401 is convenient to be taken out to be cleaned and replaced, and easy for the user to make contact with straw using hand or finger. The tube 1401 can be thrown away and replaced when damaged.

Following are some of the benefits which may be expected from using the beverage ionizing device (see http://kiflow.com/info/ions.html):
Recovery from physical exhaustion or fatigue—by increasing oxygen levels in the blood and facilitating more efficient oxygen utilization, negative ions can help accelerate recovery from fatigue
Stabilizing brain function—by promoting abundant oxygen levels in the blood, negative ions can help normalize brain function, resulting in relaxation and calmness
Blood purification—by increasing the levels of calcium and sodium in the blood stream, negative ions can help restore a healthy (slightly alkaline) pH balance to the blood
Increasing metabolism—by stimulating the exchange of electronic substances in cell walls, negative ions can help increase metabolism
Strengthening the immune system—high levels of negative ions may promote production of globulin in the blood, resulting in stronger resistance to illness
Balancing the autonomic nervous system—negative ions can calm and relax taut nerves by balancing the opposing sympathetic and parasympathetic branches of the autonomic nervous system
Promoting better digestion—by counteracting over-arousal of the sympathetic nervous system, negative ions may belp ease tension in the stomach and intestines, promoting the production of digestive enzymes and enhancing digestion
Cell rejuvenation—negative ions may belp revitalize cell metabolism, enhancing the vitality of muscle tissue and strengthening internal organs. Because the loop is in the body, very low current flows in the body, which may increase cell rejuvenation and increase metabolism.
For overall health—negative ions can rejuvenate and revitalize all of the body's systems and cells, promoting a stronger overall constitution.

Other embodiments are within the scope of the following claims.

What is claimed is:

1. A hair brushing or combing device, comprising:
a hair brush or comb body having a handle on a first end and brush bristles on a second end;
an electronic circuit substantially within the handle and having a positive terminal and a negative terminal;
a conductive anode in the handle and exposed on an outside of the handle the conductive anode coupled to the positive terminal;.
wherein the negative terminal is coupled to the bristles.

2. A nasal washing device, comprising:
a nasal wash body having a container and a cap, the cap including an opening;
a conductive anode exposed on an outside of the container;
a cathode inside the container;
an electronic circuit substantially located on the outside of the container, and having a positive terminal and a negative terminal,
wherein the negative terminal is coupled to the cathode and the positive terminal is coupled to the conductive anode.

3. A beverage ionizing device, comprising:
an apparatus that contacts liquid when a user is drinking the liquid;
a metal anode exposed on an outside of the apparatus, the metal anode being touched by the user;
a cathode inside the apparatus and contacting with the liquid;
an electronic circuit;
a switch operable to provide electrical potential to the metal anode, the switch also operable to cause activation of an ionic function.
